(19) United States
(12) Patent Application Publication
(10) Pub. No.: US 2014/0369947 A1
(43) Pub. Date: Dec. 18, 2014

(54) COSMETIC COMPOSITION COMPRISING AN ANIONIC OR NONIONIC ASSOCIATIVE POLYMER, FIXING POLYMER AND A PARTICULAR SURFACTANT

(71) Applicant: L'OREAL, Paris (FR)

(72) Inventors: Gregory Plos, Paris (FR); Estelle Mathonneau, Paris (FR); Julien Cabourg, Combs-la-Ville (FR); Laetitia Feuillette, Paris (FR)

(21) Appl. No.: 14/366,806

(22) PCT Filed: Dec. 19, 2012
(86) PCT No.: PCT/EP2012/076142

§ 371 Date: Jun. 19, 2014

Related U.S. Application Data
(60) Provisional application No. 61/592,982, filed on Jan. 31, 2012.

(30) Foreign Application Priority Data
Dec. 19, 2011 (FR) 1161944

(51) Int. Cl. A61K 8/81; A61Q 5/06

(52) U.S. Cl. CPC A61K 8/8182; A61K 8/8152; A61Q 5/06; A61K 2800/54
USPC 424/70.15; 424/70.16

(57) ABSTRACT

The present invention relates to a cosmetic composition, especially a hair composition, comprising at least one anionic or nonionic associative polymer, at least one fixing polymer and at least one nonionic surfactant comprising predominantly fatty chains having from 8 to 14 carbon atoms, the weight content of nonionic surfactants comprising predominantly fatty chains having from 8 to 14 carbon atoms in the composition being greater than or equal to 2%.

The present invention relates to a cosmetic composition for treating keratin fibres, in particular human keratin fibres such as the hair, comprising at least one anionic or nonionic associative polymer, at least one fixing polymer and at least one particular nonionic surfactant, to a use of this composition for treating hair, and also to a cosmetic treatment method using it.

In the field of styling, in particular among the hair products intended for shaping and/or for form retention of the hairstyle, the hair compositions are generally constituted of a solution, usually an alcoholic or aqueous solution, and of one or more fixing polymers as a mixture with various cosmetic adjuvants.

These compositions may be in the form of hair gels, lotions, mousses or sprays that are generally applied to wet hair before blow drying or drying.

In particular, hair gels are especially constituted of one or more thickening polymers or gelling agents in combination with one or more fixing polymers, which usually have the role of of forming a film on the surface of the keratin fibres to be fixed in order to produce welds between them, thus structuring or constructing the hairstyle and giving it hold.

Document US 2007/0134191 describes for example several styling gel compositions, one of the compositions comprising a fixing polymer, a nonionic amphiphilic polymer and an emulsifier, in particular isoceteth-20 at 4%. Document WO 2011/062805 also describes examples of a styling gel composition comprising a fixing polymer combined with an anionic associative polymer, one of the compositions comprising 0.25% of laureth-30.

Styling gels generally have medium to very strong hair fixing properties. In this fixing range, the shape-retention of the hairstyle over time is often a function of the initial fixing level of the gel, but in any case remains very inadequate. Indeed, the locks of hair obtained by the application of a gel are subjected to mechanical stresses throughout the day, such as helmet wearing, hands being run through the hair, wind, removal of clothing, etc.

Moreover, styling gels are often difficult to remove from the hair, and especially from the hands at the time of application. Soap or shampoo are systematically needed in order to completely cleanse the hands and the hair.

There is thus a real need to find cosmetic compositions, especially for styling, which allow a long-lasting form retention of the hairstyle and which are easily removed by washing.

Surprisingly and advantageously, the applicant has just discovered that by combining a particular surfactant in particular proportions with an anionic or nonionic associative polymer and a fixing polymer, a styling gel was obtained that had an improved hold of the hairstyle over time and that the gel was easily removed from the hands and the hair with water, without shampoo or soap.

One particular subject of the present invention is therefore a cosmetic composition, especially a hair composition, comprising at least one anionic or nonionic associative polymer, at least one fixing polymer and at least one nonionic surfactant comprising predominantly fatty chains having from 8 to 14 carbon atoms, the weight content of nonionic surfactants comprising predominantly fatty chains having from 8 to 14 carbon atoms in the composition being greater than or equal to 2%.

The combination according to the invention indeed makes it possible to obtain a styling gel with very strong fixation, having an extreme hold over time and with respect to mechanical stresses, especially helmet wearing or hands being run through the hair, and enables the hands and the hair to be clean after rinsing with water.

According to another of its aspects, the present invention relates to a method for treating keratin fibres, especially for shaping the hair, in which, applied to the keratin fibres, is a composition comprising at least one anionic or nonionic associative polymer, at least one fixing polymer and at least one nonionic surfactant comprising predominantly alkyl chains having from 8 to 14 carbons, the weight content of nonionic surfactants comprising predominantly alkyl chains having from 8 to 14 carbons in the composition being greater than or equal to 2%.

According to another aspect, the present invention relates to the use, for treating keratin fibres, especially for form retention and/or fixing of the hair, of a cosmetic composition as defined above.

The expression “at least one” is equivalent to the expression “one or more”.

The expression “hair composition” is understood to mean a composition that is applied to the hair, i.e. especially for form retention and/or fixing of the hairstyle, caring for the hair, making up the hair or dyeing the hair.

The expression “styling composition” is understood to mean a composition that makes it possible to give shape to a head of hair and/or to retain the acquired shape of the head of hair.

For the purposes of the present invention, the expression “fixing polymer” is thus understood to mean a polymer capable of giving shape to a head of hair and/or of retaining the acquired shape of the head of hair.

The expression “associative polymers” is understood, for the purposes of the invention, to mean amphiphilic polymers comprising both a) one or more hydrophobic units each constituted by one or more fatty chains and b) one or more hydrophilic units. These polymers result from a polymerization involving at least one monomer other than an alkylene oxide or glycidol oxide. They may, for example, be obtained by radical polymerization reactions, polycondensation reactions, and grafting reactions on prepolymers.

The expression “fatty chain” should be understood according to the invention to mean an optionally hydroxylated, linear or branched alkyl or alkenyl chain having from 8 to 30, and preferably from 10 to 30, carbon atoms.

The associative polymers of the composition according to the invention are of anionic or nonionic type.

Among the associative polymers of anionic type that may be mentioned are:
(I) those comprising at least one hydrophilic unit, and at least one fatty-chain allyl ether unit, more particularly those for which the hydrophilic unit is constituted of an ethylenically unsaturated anionic monomer, more particularly still of a vinylcarboxylic acid and very particularly of an acrylic acid or a methacrylic acid or mixtures thereof, and the fatty-chain allyl ether unit of which corresponds to the monomer of formula (I) below:

CH₂═CR′CH₂OBₙR (I)

in which R′ denotes H or CH₃, B denotes an ethylenoxy radical, n is zero or denotes an integer ranging from 1 to 100, R denotes a hydrocarbon-based radical chosen from alkyl, arylalkyl, aryl, alkylaryl and cycloalkyl radicals, comprising from 8 to 30 carbon atoms, preferably 10 to 24 and more particularly still from 12 to 18 carbon atoms. A unit of formula (I) that is more particularly preferred is a unit in which R′ denotes H, n is equal to 10, and R denotes a stearyl radical (C₁₈).

Anionic associative polymers of this type are described and prepared, according to an emulsion polymerization process, in patent EP-0 216 479.

Among these anionic associative polymers, those that are particularly preferred according to the invention are polymers formed from 20% to 60% by weight of acrylic acid and/or of methacrylic acid, from 5% to 60% by weight of C₁-C₄ alkyl (meth)acrylates, from 2% to 50% by weight of fatty-chain allyl ether of formula (I), and from 0 to 1% by weight of a crosslinking agent which is a well-known copolymerizable unsaturated polyethylenic monomer, such as diallyl phthalate, allyl (meth)acrylate, divinylbenzene, (poly)ethylene glycol dimethacrylate or methylenebisacrylamide.

Among the latter polymers, those that are very particularly preferred are crosslinked terpolymers of methacrylic acid, ethyl acrylate and polyethylene glycol (10 EO) stearyl alcohol ether (Steareth-10), in particular those sold by the company Ciba under the names Salcare SC 80® and Salcare SC 90®, which are aqueous 30% emulsions of a crosslinked terpolymer of methacrylic acid, ethyl acrylate and steareth-10 allyl ether (40/50/10);
(II) those comprising at least one hydrophilic unit of unsaturated olefinic carboxylic acid type, and at least one hydrophobic unit of (C₁₀-C₃₀)alkyl ester of unsaturated carboxylic acid type.

These polymers are preferably chosen from those in which the hydrophilic unit of unsaturated olefinic carboxylic acid type corresponds to the monomer of formula (II) below:

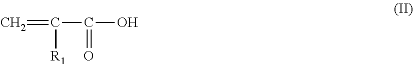

in which R₁ denotes H or CH₃ or C₂H₅, i.e. acrylic acid, methacrylic acid or ethacrylic acid units, and the hydrophobic unit of which of (C₁₀-C₃₀) alkyl ester of unsaturated carboxylic acid type corresponds to the monomer of formula (III) below:

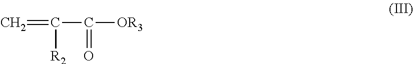

in which R₂ denotes H or CH₃ or C₂H₅ (i.e. acrylate, methacrylate or ethacrylate units) and preferably H (acrylate units) or CH₃ (methacrylate units), R₃ denoting a C₁₀-C₃₀ and preferably C₁₂-C₂₂ alkyl radical.

(C₁₀-C₃₀) alkyl esters of unsaturated carboxylic acids according to the invention include, for example, lauryl acrylate, stearyl acrylate, decyl acrylate, isodecyl acrylate and dodecyl acrylate, and the corresponding methacrylates, lauryl methacrylate, stearyl methacrylate, decyl methacrylate, isodecyl methacrylate and dodecyl methacrylate.

Anionic polymers of this type are described and prepared, for example, according to patents U.S. Pat. No. 3,915,921 and U.S. Pat. No. 4,509,949.

Among anionic associative polymers of this type, use will more particularly be made of polymers formed from a monomer mixture comprising:

(i) essentially acrylic acid,

(ii) an ester of formula (III) described above in which R₂ denotes H or CH₃, R₃ denoting an alkyl radical containing from 12 to 22 carbon atoms,

(iii) and a crosslinking agent, which is a well-known copolymerizable polyethylenically unsaturated monomer, such as diallyl phthalate, allyl (meth)acrylate, divinylbenzene, (poly)ethylene glycol dimethacrylate or methyl enebisacrylamide.

Among anionic associative polymers of this type, use will more particularly be made of those constituted of from 95% to 60% by weight of acrylic acid (hydrophilic unit), 4% to 40% by weight of C₁₀-C₃₀ alkyl acrylate (hydrophobic unit) and 0 to 6% by weight of crosslinking polymerizable monomer, or alternatively those constituted of from 98% to 96% by weight of acrylic acid (hydrophilic unit), 1% to 4% by weight of C₁₀-C₃₀ alkyl acrylate (hydrophobic unit) and 0.1% to 0.6% by weight of crosslinking polymerizable monomer such as those described above.

Among said above polymers, those very particularly preferred according to the present invention are the products sold by the company Lubrizol under the trade names Pemulen TR1®, Pemulen TR20 and Carbopol 1382®, and more preferably still Pemulen TR1®, and the product sold by the company SEPPIC under the name Coatex SX®.
(III) maleic anhydride/C₃₀-C₃₅ α-olefin/alkyl maleate terpolymers, such as the product (maleic anhydride/C₃₀-C₃₅ α-olefin/isopropyl maleate copolymer) sold under the name Performa V 1608® by the company Newphase Technologies.
(IV) acrylic terpolymers comprising:

(a) around 20% to 70% by weight of an α,β-monoethylenically unsaturated carboxylic acid,

(b) around 20% to 80% by weight of a non-surfactant α,β-monoethylenically unsaturated monomer different from (a),

(c) around 0.5% to 60% by weight of a nonionic monourethane which is the reaction product of a monohydric surfactant with a monoethylenically unsaturated monoisocyanate,

such as those described in patent application EP-A-0 173 109 and more particularly the terpolymer described in Example 3, namely a methacrylic acid/methyl acrylate/ethoxylated (40 EO) behenyl alcohol dimethyl-meta-isopropenyl benzyl isocyanate terpolymer, as an aqueous 25% dispersion.
(V) copolymers comprising among their monomers an α,β-monoethylenically unsaturated carboxylic acid and an ester of an α,β-monoethylenically unsaturated carboxylic acid and of an oxyalkylenated fatty alcohol.

Preferably, these compounds also comprise, as monomer, an ester of an α,β-monoethylenically unsaturated carboxylic acid and of a C₁-C₄ alcohol.

By way of example of a compound of this type, mention may be made of Aculyn 22® and Aculyn 88® sold by the company Rohm & Haas, which are methacrylic acid/ethyl acrylate/oxyalkylenated stearyl methacrylate terpolymers.

Mention may also be made of Synthalen W400 sold by the company 3V-Sigma, which is an acrylate copolymer as an aqueous emulsion, and Synthalen W2000 sold by the company 3V-Sigma, which is an acrylate/polyoxyethylenated (25 EO) C₁₂-C₂₄ alkyl acrylate copolymer as an aqueous emulsion.

Mention may also be made of the product Structure 2001 sold by the company Akzo Nobel, which is an acrylic acid/oxyethylenated (20 EO) monostearyl itaconate copolymer as an aqueous 30% dispersion.

Mention will also be made of the products sold by the company Lubrizol under the commercial references Carbopol Ultrez 21, which is an acrylic polymer (acrylates/C₁₀-C₃₀ alkyl acrylate crosspolymer) and Novethix L-10, which is an acrylates/beheneth-25 methacrylate copolymer.

According to the invention, the associative polymers of nonionic type are preferably selected from:
(1) celluloses modified with groups comprising at least one fatty chain. Examples that may be mentioned include:
hydroxyethyl celluloses modified with groups comprising at least one fatty chain, such as alkyl, arylalkyl or alkylaryl groups, or mixtures thereof, and in which the alkyl groups are preferably C₈-C₂₂ alkyl groups, such as the product Natrosol Plus Grade 330 CS® (C₁₆ alkyls) sold by the company Aqualon, or the product Bermocoll EHM 100C) sold by the company Berol Nobel,
hydroxyethyl celluloses modified with alkylphenyl polyalkylene glycol ether groups, such as the product Amercell Polymer HM-1500® (nonylphenyl polyethylene glycol (15) ether) sold by the company Amerchol.
(2) hydroxypropyl guars modified with groups comprising at least one fatty chain, such as the product Esaflor HM 22® (C₂₂ alkyl chain) sold by the company Lamberti, and the products RE210-18® (C₁₄ alkyl chain) and RE205-1® (C₂₀ alkyl chain) sold by the company Rhodia Chimie.
(3) inulins modified with groups comprising at least one fatty chain, such as alkyl carbamate inulins and in particular the lauryl carbamate inulin sold by the company Orafti under the name Inutec SP1.
(4) copolymers of vinylpyrrolidone and of fatty-chain hydrophobic monomers; examples that may be mentioned include:
the products Antaron V216® or Ganex V216® (vinylpyrrolidone/hexadecene copolymer) sold by the company I.S.P.
the products Antaron V220® or Ganex V220® (vinylpyrrolidone/eicosene copolymer) sold by the company I.S.P.
(5) copolymers of C₁-C₆ alkyl methacrylates or acrylates and of amphiphilic monomers comprising at least one fatty chain, such as, for example, the oxyethylenated methyl acrylate/stearyl acrylate copolymer sold by the company Goldschmidt under the name Antil 208®.
(6) copolymers of hydrophilic methacrylates or acrylates and of hydrophobic monomers comprising at least one fatty chain, such as, for example, the polyethylene glycol methacrylate/lauryl methacrylate copolymer.
(7) polyether polyurethanes comprising in their chain both hydrophilic blocks usually of polyoxyethylenated nature and hydrophobic blocks, which may be aliphatic sequences alone and/or cycloaliphatic and/or aromatic sequences.
(8) polymers with an aminoplast ether backbone containing at least one fatty chain, such as the Pure Thix® compounds sold by the company Sud-Chemie.

Preferably, the polyether polyurethanes comprise at least two hydrocarbon-based lipophilic chains containing from 6 to 30 carbon atoms, separated by a hydrophilic block, the hydrocarbon-based chains possibly being pendent chains or chains at the end of the hydrophilic block. In particular, it is possible for one or more pendent chains to be provided. In addition, the polymer may comprise a hydrocarbon-based chain at one end or at both ends of a hydrophilic block.

The polyether polyurethanes may be multiblock, in particular in triblock form. The hydrophobic blocks may be at each end of the chain (for example: triblock copolymer containing a hydrophilic central block) or distributed both at the ends and in the chain (for example multiblock copolymer). These same polymers may also be graft polymers or star polymers.

The nonionic fatty-chain polyether polyurethanes may be triblock copolymers in which the hydrophilic block is a polyoxyethylenated chain comprising from 50 to 1000 oxyethylene groups. The nonionic polyether polyurethanes comprise a urethane bond between the hydrophilic blocks, whence arises the name.

By extension, also included among the nonionic fatty-chain polyether polyurethanes are those in which the hydrophilic blocks are linked to the lipophilic blocks via other chemical bonds.

As examples of nonionic fatty-chain polyether polyurethanes that may be used in the invention, it is also possible to use Rheolate FX 1100 (Steareth-100/PEG 136/HDI copolymer), Rheolate 205® containing a urea function, sold by the company Elementis, or else Rheolate® 208, 204 or 212, and also Acrysol RM 184®.

Mention may also be made of the product Elfacos T210® containing a C₁₂-C₁₄ alkyl chain, and the product Elfacos T212® containing a C₁₈ alkyl chain, from Akzo.

The product DW 1206B® from Röhm & Haas containing a C₂₀ alkyl chain and a urethane bond, sold at a solids content of 20% in water, may also be used

It is also possible to use solutions or dispersions of these polymers, in particular in water or in aqueous-alcoholic medium. Examples of such polymers that may be mentioned are Rheolate® 255, Rheolate® 278 and Rheolate® 244 sold by the company Elementis. The products DW 1206F and DW 1206J sold by the company Röhm & Haas may also be used.

The polyether polyurethanes that may be used according to the invention are in particular those described in the article by G. Fonnum, J. Bakke and Fk. Hansen—Colloid Polym. Sci., 271, 380-389 (1993).

More particularly still, according to the invention, it is also possible to use a polyether polyurethane that may be obtained by polycondensation of at least three compounds comprising (i) at least one polyethylene glycol comprising from 150 to 180 mol of ethylene oxide, (ii) stearyl alcohol or decyl alcohol, and (iii) at least one diisocyanate.

Such polyether polyurethanes are sold especially by the company Rohm & Haas under the names Aculyn 46® and Aculyn 44® [Aculyn 46® is a polycondensate of polyethylene glycol containing 150 or 180 mol of ethylene oxide, of stearyl alcohol and of methylenebis(4-cyclohexyl isocyanate) (SMDI), at 15% by weight in a matrix of maltodextrin (4%) and water (81%); Aculyn 44® is a polycondensate of polyethylene glycol containing 150 or 180 mol of ethylene oxide, of decyl alcohol and of methylenebis(4-cyclohexyl isocyanate) (SMDI), at 35% by weight in a mixture of propylene glycol (39%) and water (26%)].

The associative polymer(s) of the composition is (are) preferably present in a concentration ranging from 0.01% to 30% by weight, better still in a concentration ranging from 0.1% to 20% by weight and more preferably still ranging from 0.5% to 10% by weight of the total weight of the composition.

The surfactants of the composition according to the invention are of nonionic type and comprise predominantly fatty chains having from 8 to 14 carbon atoms.

The term “surfactants” is understood, for the purposes of the present invention, to mean any compound that reduces the surface tension of a liquid and in particular of water at 25° C., the chemical structure of which a) does not involve a polymerization reaction or b) involves only the sole polymerization of an alkylene oxide and/or glycidol oxide.

The expression “fatty chain” should be understood according to the invention to mean, as for associative polymers, an optionally hydroxylated, linear or branched alkyl or alkenyl chain having from 8 to 30, and preferably from 10 to 30, carbon atoms.

The nonionic surfactants are often mixtures of compounds having, in particular, different lengths of alkyl fatty chains.

The term “predominantly” is then understood to mean that, for the surfactant in question, the weight percentage of compounds having fatty chains ranging from C₈ to C₁₄ is greater than or equal to 50%.

In other words, the nonionic surfactants that can be used in the composition of the invention comprise a mixture of compounds having fatty chains of different lengths, which mixture comprises more than 50% of compounds having fatty chains ranging from C₈ to C₁₄.

Examples of nonionic surfactants that can be used in the composition used according to the invention are described, for example, in the “Handbook of Surfactants” by M. R. Porter, published by Blackie & Son (Glasgow and London), 1991, pp. 116-178. They are especially chosen from alcohols, α-diols and (C₁-C₂₀)alkylphenols, these compounds being polyethoxylated, polypropoxylated and/or polyglycerolated, and containing at least one fatty chain comprising, for example, predominantly from 8 to 14 carbon atoms, it being possible for the number of ethylene oxide and/or propylene oxide groups to range in particular from 1 to 100, and for the number of glycerol groups to range in particular from 2 to 30.

Mention may also be made of copolymers of ethylene oxide and propylene oxide, optionally oxyethylenated sorbitan fatty acid esters, sucrose fatty acid esters, polyoxyalkylenated fatty acid esters, optionally oxyalkylenated alkyl polyglycosides, alkyl glucoside esters, derivatives of N-alkylglucamine and of N-acylmethylglucamine, aldobionamides and amine oxides, the fatty chains of these compounds having to be predominantly C₈-C₁₄ fatty chains.

Mention may be made, as examples of oxyalkylenated nonionic surfactants, of:

oxyalkylenated (C₈-C₁₄)alkylphenols;

oxyalkylenated linear or branched, saturated or unsaturated C₈-C₁₄ alcohols;

oxyalkylenated linear or branched, saturated or unsaturated C₈-C₁₄ amides;

esters of linear or branched, saturated or unsaturated C₈-C₁₄ acids and polyethylene glycols;

polyoxyethylenated esters of linear or branched, saturated or unsaturated C₈-C₁₄ acids and sorbitol;

saturated or unsaturated oxyethylenated vegetable oils.

The surfactants contain a number of moles of ethylene oxide and/or of propylene oxide of between 1 and 100, preferably between 2 and 50 and preferably between 2 and 30. Advantageously, the nonionic surfactants do not comprise oxypropylene units.

As examples of monoglycerolated or polyglycerolated nonionic surfactants, use is preferably made of monoglycerolated or polyglycerolated C₈-C₁₄ alcohols.

In particular, the monoglycerolated or polyglycerolated C₈-C₁₄ alcohols correspond to formula (A7) below:

R₂₉O—[CH₂—CH(CH₂OH)—O]ₘ—H (A7)

in which formula (A7):

R₂₉ represents an optionally hydroxylated, linear or branched C₈-C₁₄ alkyl or alkenyl radical; and

m represents a number ranging from 1 to 30 and preferably from 1 to 10.

In accordance with a preferred embodiment of the invention, the nonionic surfactants are selected from oxyethylenated C₈-C₁₄ alcohols comprising from 1 to 100 mol of ethylene oxide, polyoxyethylenated esters of linear or branched, saturated or unsaturated C₈-C₁₄ acids and of sorbitol comprising from 1 to 100 mol of ethylene oxide, C₈-C₁₄ alkyl polyglycosides, monoglycerolated or polyglycerolated nonionic surfactants of C₈-C₁₄ alcohols; which are optionally hydroxylated.

As examples of compounds of formula (A7) that are suitable within the context of the invention, mention may be made of lauryl alcohol containing 4 mol of glycerol (INCI name: Polyglyceryl-4 Lauryl Ether), lauryl alcohol containing 1.5 mol of glycerol, oleyl alcohol containing 4 mol of glycerol (INCI name: Polyglyceryl-4 Oleyl Ether), oleyl alcohol containing 2 mol of glycerol (INCI name: Polyglyceryl-2 Oleyl Ether), cetearyl alcohol containing 2 mol of glycerol, cetearyl alcohol containing 6 mol of glycerol, oleocetyl alcohol containing 6 mol of glycerol, and octadecanol containing 6 mol of glycerol.

The alcohol of formula (A7) may represent a mixture of alcohols in the same way that the value of m represents a statistical value, which means that, in a commercial product, several species of polyglycerolated fatty alcohols may coexist in the form of a mixture.

Within the context of the invention use may especially be made, as nonionic surfactant, of the products that correspond to the INCI names Laureth-4, Laureth-6, polysorbate 20, coco-glucoside and polyglyceryl-3-hydroxylauryl ether.

The nonionic surfactant(s) comprising predominantly fatty chains having from 8 to 14 carbon atoms of the present invention is (are) present in a concentration greater than or equal to 2% by weight, preferably in a concentration ranging from 2% to 40% by weight, preferentially ranging from 2% to 25% by weight and more preferentially still ranging from 4% to 10% by weight of the total weight of the composition.

All the anionic, cationic, amphoteric and nonionic fixing polymers and mixtures thereof used in the art may be used in the compositions according to the present application.

The fixing polymers may be soluble in the composition or insoluble in this same composition and may be used in this case in the form of dispersions of solid or liquid particles of polymer (latex or pseudo-latex).

The anionic fixing polymers generally used are polymers comprising groups derived from carboxylic acid, sulfonic acid or phosphoric acid and have a number-average molecular weight of between approximately 500 and 5 000 000.

The anionic fixing polymers containing carboxylic groups that are preferred according to the invention are:

A) copolymers of acrylic acid and of acrylamide sold in the form of their sodium salts under the names Reten 421, 423 or 425 by the company Hercules, the sodium salts of polyhydroxycarboxylic acids;

B) copolymers of acrylic or methacrylic acid with a monoethylenic monomer such as ethylene, styrene, vinyl esters, acrylic or methacrylic acid esters, optionally grafted onto a polyalkylene glycol such as polyethylene glycol and optionally crosslinked. Such polymers are described in particular in French patent 1 222 944 and German patent application 2 330 956, the copolymers of this type comprising an optionally N-alkylated and/or hydroxyalkylated acrylamide unit in their chain as described in particular in Luxembourg patent applications 75370 and 75371 or sold under the name Quadramer by the company American Cyanamid. Mention may also be made of the acrylic acid/ethyl acrylate/N-tert-butylacrylamide terpolymers such as Ultrahold Strong sold by the company BASF. Mention may also be made of copolymers of acrylic acid and of C₁-C₄ alkyl methacrylate and terpolymers of vinylpyrrolidone, of acrylic acid and of C₁-C₂₀ alkyl, for example lauryl, methacrylate, such as the product sold by the company ISP under the name Acrylidone® LM and methacrylic acid/ethyl acrylate/tert-butyl acrylate terpolymers such as the product sold under the name Luvimer® 100 P by the company BASF. Mention may also be made of acrylate/hydroxyester acrylate copolymers, such as Acudyne 180 sold by the company Dow Chemical.

Mention may also be made of methacrylic acid/acrylic acid/ethyl acrylate/methyl methacrylate copolymers as an aqueous dispersion, sold under the name Amerhold® DR 25 by the company Amerchol;

C) copolymers of crotonic acid, such as those comprising vinyl acetate or propionate units in their chain and optionally other monomers such as allyl esters or methallyl esters, vinyl ether or vinyl ester of a linear or branched saturated carboxylic acid with a long hydrocarbon-based chain, such as those containing at least 5 carbon atoms, it being possible for these polymers optionally to be grafted or crosslinked, or alternatively another vinyl, allyl or methallyl ester monomer of an α- or β-cyclic carboxylic acid. Such polymers are described, inter alia, in French patents 1 222 944, 1 580 545, 2 265 782, 2 265 781, 1 564 110 and 2 439 798. Commercial products that fall within this category are the resins 28-29-30, 26-13-14 and 28-13-10, sold by the company Akzo Nobel;

D) copolymers of C₄-C₈ monounsaturated carboxylic acids or anhydrides chosen from:
copolymers comprising (i) one or more maleic, fumaric or itaconic acids or anhydrides and (ii) at least one monomer chosen from vinyl esters, vinyl ethers, vinyl halides, phenylvinyl derivatives, acrylic acid and its esters, the anhydride functions of these copolymers optionally being monoesterified or monoamidated. Such polymers are described, in particular, in U.S. Pat. Nos. 2,047,398, 2,723,248 and 2,102,113, and GB patent 839 805. Commercial products are especially those sold under the names Gantrez® AN or ES by the company ISP;
copolymers comprising (i) one or more maleic, citraconic or itaconic anhydride units and (ii) one or more monomers selected from allyl or methallyl esters optionally comprising one or more acrylamide, methacrylamide, α-olefin, acrylic or methacrylic ester, acrylic or methacrylic acid or vinylpyrrolidone groups in their chain,
   the anhydride functions of these copolymers optionally being monoesterified or monoamidated.

These polymers are described, for example, in French patents 2 350 384 and 2 357 241 by the applicant,

E) polyacrylamides comprising carboxylate groups;

F) homopolymers and copolymers comprising sulfonic groups such as polymers comprising vinylsulfonic, styrenesulfonic, naphthalenesulfonic or acrylamidoalkylsulfonic units.

These polymers can be chosen in particular from:
polyvinylsulfonic acid salts having a molecular weight of approximately between 1000 and 100 000, and also the copolymers with an unsaturated comonomer such as acrylic or methacrylic acids and their esters, and also acrylamide or its derivatives, vinyl ethers and vinylpyrrolidone;
polystyrenesulfonic acid salts, such as the sodium salts sold, for example, under the names Flexan® 500 and Flexan® 130 by Akzo Nobel; these compounds are described in patent FR 2 198 719;
polyacrylamidesulfonic acid salts, such as those mentioned in U.S. Pat. No. 4,128,631 and more particularly polyacrylamidoethylpropanesulfonic acid sold under the name Cosmedia Polymer HSP 1180 by Henkel.

As another anionic fixing polymer that can be used according to the invention, mention may be made of the branched block anionic polymer sold under the name Fixate G-100 by the company Lubrizol.

According to the invention, the anionic fixing polymers are preferably chosen from copolymers of acrylic acid or of acrylic esters, such as the acrylic acid/ethyl acrylate/N-tert-butylacrylamide terpolymers sold especially under the name Ultrahold® Strong by the company BASF, copolymers derived from crotonic acid, such as vinyl acetate/vinyl tert-butylbenzoate/crotonic acid terpolymers and the crotonic acid/vinyl acetate/vinyl neododecanoate terpolymers sold especially under the name Resin 28-29-30 by the company

Akzo Nobel, polymers derived from maleic, fumaric or itaconic acids or anhydrides with vinyl esters, vinyl ethers, vinyl halides, phenylvinyl derivatives and acrylic acid and esters thereof, such as the methyl vinyl ether/monoesterified maleic anhydride copolymers sold, for example, under the name Gantrez® by the company ISP, the copolymers of methacrylic acid and of methyl methacrylate sold under the name Eudragit® L by the company Rohm Pharma, the copolymers of methacrylic acid and of ethyl acrylate sold under the name Luvimer® MAEX or MAE by the company BASF, the vinyl acetate/crotonic acid copolymers sold under the name Luviset CA 66 by the company BASF, the vinyl acetate/crotonic acid copolymers grafted with polyethylene glycol sold under the name Aristoflex® A by the company BASF, and the polymer sold under the name Fixate G-100 L by the company Lubrizol.

The cationic fixing film-forming polymers that can be used according to the present invention are preferably selected from polymers comprising primary, secondary, tertiary and/or quaternary amine groups forming part of the polymer chain or directly attached thereto, and having a molecular weight of between 500 and about 5 000 000 and preferably between 1000 and 3 000 000.

Among these polymers, mention may be made more particularly of the following cationic polymers:

(1) homopolymers or copolymers derived from acrylic or methacrylic esters or amides and comprising at least one of the units of the following formulae:

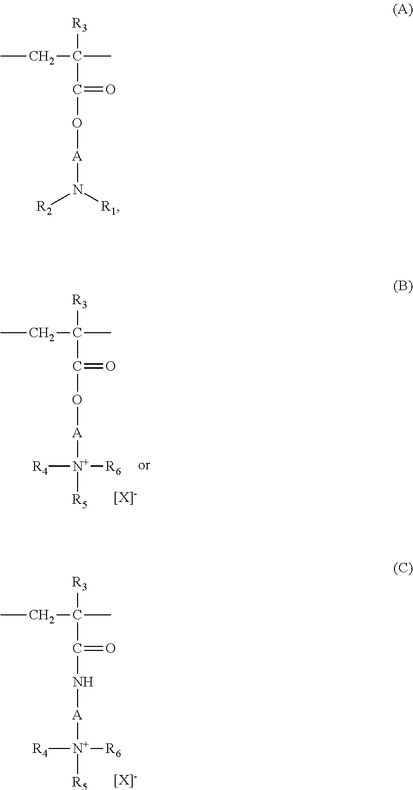

in which:
R₃ denotes a hydrogen atom or a CH₃ radical;
A is a linear or branched alkyl group comprising from 1 to 6 carbon atoms or a hydroxyalkyl group comprising from 1 to 4 carbon atoms;
R₄, R₅ and R₆, which may be identical or different, represent an alkyl group having from 1 to 18 carbon atoms or a benzyl radical;
R₁ and R₂, which may be identical or different, each represent a hydrogen atom or an alkyl group having from 1 to 6 carbon atoms;
X denotes a methosulfate anion or a halide such as chloride or bromide.

The copolymers of the family (1) also contain one or more units derived from comonomers that may be selected from the family of acrylamides, methacrylamides, diacetone acrylamides, acrylamides and methacrylamides substituted on the nitrogen with lower (C₁-C₄) alkyl groups, groups derived from acrylic or methacrylic acids or esters thereof, vinyllactams such as vinylpyrrolidone or vinylcaprolactam, and vinyl esters.

Thus, among these copolymers of the family (1), mention may be made of:
copolymers of acrylamide and of dimethylaminoethyl methacrylate quaternized with dimethyl sulfate or with a dimethyl halide, such as the product sold under the name Hercofloc® by the company Hercules,
copolymers of acrylamide and of methacryloyloxyethyltrimethylammonium chloride, described, for example, in patent application EP-A-080 976 and sold under the name Bina Quat P 100 by the company Ciba Geigy,
the copolymer of acrylamide and of methacryloyloxyethyltrimethylammonium methosulfate, such as the product sold under the name Reten by the company Hercules,
quaternized or non-quaternized vinylpyrrolidone/dialkylaminoalkyl acrylate or methacrylate copolymers, such as the products sold under the name Gafquat® by the company ISP, such as, for example, Gafquat® 734 or Gafquat® 755, or alternatively the products known as Copolymer® 845, 958 and 937. These polymers are described in detail in French patents 2 077 143 and 2 393 573,
fatty-chain polymers containing a vinylpyrrolidone unit, such as the products sold under the name Styleze W20 and Styleze W10 by the company ISP,
dimethylaminoethyl methacrylate/vinylcaprolactam/vinylpyrrolidone terpolymers, such as the product sold under the name Gaffix VC 713 by the company ISP, and
quaternized vinylpyrrolidone/dimethylaminopropylmethacrylamide copolymers, such as the products sold under the name Gafquat® HS 100 by the company ISP;

(2) non-cellulose cationic polysaccharides, preferably containing quaternary ammonium, such as those described in U.S. Pat. Nos. 3,589,578 and 4,031,307, such as guar gums containing trialkylammonium cationic groups. Such products are sold in particular under the trade names Jaguar C13 S, Jaguar C 15 and Jaguar C 17 by the company Meyhall;

(3) quaternary copolymers of vinylpyrrolidone and of vinylimidazole;

(4) chitosans or salts thereof; the salts that can be used are, in particular, chitosan acetate, lactate, glutamate, gluconate or pyrrolidonecarboxylate.

Among these compounds, mention may be made of chitosan having a degree of deacetylation of 90.5% by weight, sold under the name Kytan Brut Standard by the company Aber Technologies, and chitosan pyrrolidonecarboxylate sold under the name Kytamer® PC by the company Amerchol;

(5) cationic cellulose derivatives such as copolymers of cellulose or of cellulose derivatives grafted with a water-soluble monomer comprising a quaternary ammonium, and described in particular in U.S. Pat. No. 4,131,576, such as hydroxyalkyl celluloses, for instance hydroxymethyl, hydroxyethyl or hydroxypropyl celluloses grafted in particular with a methacryloyloxyethyltrimethylammonium, methacrylamidopropyltrimethylammonium or dimethyl-diallylammonium salt.

The products sold corresponding to this definition are, more particularly, the products sold under the names Celquat L 200 and Celquat H 100 by the company Akzo Nobel;

(6) partially hydrolysed polyvinylformamide polymers, such as the products sold under the names Lupamin 9095, Lupamin 9010 and Lupamin 9030 by the company BASF.

The amphoteric fixing polymers that can be used in accordance with the invention can be chosen from polymers comprising units B and C distributed randomly in the polymer chain, where B denotes a unit derived from a monomer comprising at least one basic nitrogen atom and C denotes a unit derived from an acid monomer comprising one or more carboxylic or sulfonic groups, or alternatively B and C can denote groups derived from carboxybetaine or sulfobetaine zwitterionic monomers;

B and C can also denote a cationic polymer chain comprising primary, secondary, tertiary or quaternary amine groups, in which at least one of the amine groups bears a carboxylic or sulfonic group connected via a hydrocarbon-based group, or alternatively B and C form part of a chain of a polymer comprising an α,β-dicarboxylic ethylene unit in which one of the carboxylic groups has been made to react with a polyamine comprising one or more primary or secondary amine groups.

The amphoteric fixing polymers corresponding to the definition given above that are more particularly preferred are chosen from the following polymers:

(1) Copolymers comprising acidic vinyl units and basic vinyl units, such as those resulting from the copolymerization of a monomer derived from a vinyl compound bearing a carboxylic group such as, more particularly, acrylic acid, methacrylic acid, maleic acid, α-chloroacrylic acid, and of a basic monomer derived from a substituted vinyl compound containing at least one basic atom, such as, more particularly, dialkylaminoalkyl methacrylates and acrylates, dialkylaminoalkylmethacrylamides and acrylamides. Such compounds are described in U.S. Pat. No. 3,836,537.
(2) Polymers comprising units derived from:

a) at least one monomer selected from acrylamides and methacrylamides substituted on the nitrogen atom by an alkyl group,

b) at least one acidic comonomer containing one or more reactive carboxylic groups, and

c) at least one basic comonomer such as esters containing primary, secondary, tertiary and quaternary amine substituents of acrylic and methacrylic acids and the product of quaternization of dimethylaminoethyl methacrylate with dimethyl or diethyl sulfate.

The N-substituted acrylamides or methacrylamides that are more particularly preferred according to the invention are compounds in which the alkyl groups contain from 2 to 12 carbon atoms and more particularly N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers are chosen more particularly from acrylic, methacrylic, crotonic, itaconic, maleic and fumaric acids and alkyl monoesters, having 1 to 4 carbon atoms, of maleic or fumaric acid or anhydride.

The preferred basic comonomers are aminoethyl, butylaminoethyl, N,N′-dimethylaminoethyl and N-tert-butylaminoethyl methacrylates.

Use is particularly made of the copolymers having the CTFA (4th edition, 1991) name octylacrylamide/acrylates/butylaminoethyl methacrylate copolymer, such as the products sold under the name Amphomer® or Lovocryl® 47 by the company Akzo Nobel.

(3) Crosslinked and acylated polyaminoamides partially or totally derived from polyaminoamides of general formula:

CO—R₁₀—CO—Z (IV)

in which R₁₀ represents a divalent group derived from a saturated dicarboxylic acid, an aliphatic monocarboxylic or dicarboxylic acid containing an ethylenic double bond, an ester of a lower alkanol, having 1 to 6 carbon atoms, of these acids, or a group derived from the addition of any one of said acids to a bis(primary) or bis(secondary) amine, and Z denotes a group derived from a bis(primary), mono(secondary) or bis(secondary) polyalkylene-polyamine and preferably represents:
a) in proportions of from 60 to 100 mol %, the group:

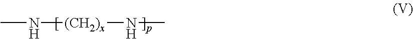

where x=2 and p=2 or 3, or alternatively x=3 and p=2
this group being derived from diethylenetriamine, from triethylenetetramine or from dipropylenetriamine;
b) in proportions of from 0 to 40 mol %, the group (V) above, in which x=2 and p=1 and which is derived from ethylenediamine, or the group derived from piperazine:

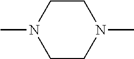

c) in proportions of from 0 to 20 mol %, the —NH(CH₂)₆—NH— group being derived from hexamethylenediamine,
these polyaminoamides being crosslinked by addition reaction of a difunctional crosslinking agent selected from epihalohydrins, diepoxides, dianhydrides and bis-unsaturated derivatives, using from 0.025 to 0.35 mol of crosslinking agent per amine group of the polyaminoamide, and acylated by the action of acrylic acid, chloroacetic acid or an alkane sultone, or salts thereof.

The saturated carboxylic acids are preferably chosen from acids having 6 to 10 carbon atoms, such as adipic acid, 2,2,4-trimethyladipic acid and 2,4,4-trimethyladipic acid, terephthalic acid, acids containing an ethylenic double bond such as, for example, acrylic acid, methacrylic acid and itaconic acid.

The alkane sultones used in the acylation are preferably propane sultone or butane sultone; the salts of the acylating agents are preferably the sodium or potassium salts.

(4) Polymers comprising zwitterionic units of formula:

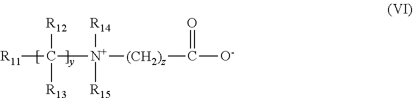

in which R₁₁ denotes a polymerizable unsaturated group such as an acrylate, methacrylate, acrylamide or methacrylamide group, y and z represent an integer from 1 to 3, R₁₂ and R₁₃ represent a hydrogen atom, a methyl, ethyl or propyl group, R₁₄ and R₁₅ represent a hydrogen atom or an alkyl group such that the sum of the carbon atoms in R₁₄ and R₁₅ does not exceed 10.

The polymers comprising such units can also comprise units derived from non-zwitterionic monomers such as dimethylaminoethyl or diethylaminoethyl acrylate or methacrylate or alkyl acrylates or methacrylates, acrylamides or methacrylamides or vinyl acetate.

Mention may be made, by way of example, of methyl methacrylate/methyl dimethylcarboxymethylammonioethyl methacrylate copolymers, such as the product sold under the name Diaformer Z301 by the company Sandoz.

(5) Polymers derived from chitosan comprising monomer units corresponding to the following formulae:

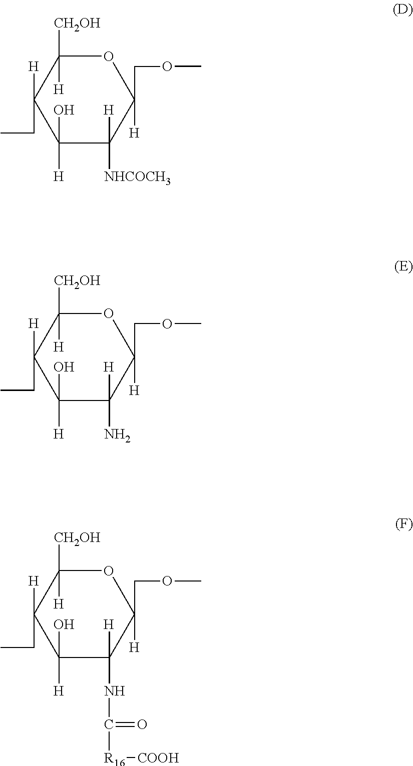

the unit (D) being present in proportions of between 0 and 30%, the unit (E) in proportions of between 5 and 50% and the unit (F) in proportions of between 30% and 90%, it being understood that, in this unit (F), R₁₆ represents a group of formula:

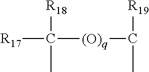

in which, if q=0, R₁₇, R₁₈ and R₁₉, which may be identical or different, each represent a hydrogen atom, a methyl, hydroxyl, acetoxy or amino residue, a monoalkylamine residue or a dialkylamine residue that are optionally interrupted by one or more nitrogen atoms and/or optionally substituted with one or more amine, hydroxyl, carboxyl, alkylthio or sulfonic groups, an alkylthio residue in which the alkyl group bears an amino residue, at least one of the groups R₁₇, R₁₈ and R₁₉ being, in this case, a hydrogen atom;
or, if q=1, R₁₇, R₁₈ and R₁₉ each represent a hydrogen atom, and also the salts formed by these compounds with bases or acids.
(6) Polymers with units corresponding to the general formula (VII) are described, for example, in French patent 1 400 366:

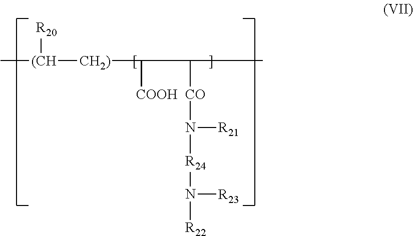

in which R₂₀ represents a hydrogen atom, a CH₃O, CH₃CH₂O or phenyl group, R₂₁ denotes a hydrogen atom or a lower alkyl group such as methyl or ethyl, R₂₂ denotes a hydrogen atom or a C₁-C₆ lower alkyl group such as methyl or ethyl, R₂₃ denotes a C₁-C₆ lower alkyl group such as methyl or ethyl or a group corresponding to the formula: —R₂₄—N(R₂₂)₂, R₂₄ representing a —CH₂—CH₂—, —CH₂—CH₂—CH₂— or —CH₂—CH(CH₃)— group, R₂₂ having the meanings mentioned above.
(7) Polymers derived from the N-carboxyalkylation of chitosan, such as N-carboxymethylchitosan or N-carboxybutylchitosan sold under the name Evalsan by the company Jan Dekker.
(8) Amphoteric polymers of the -D-X-D-X— type chosen from:
a) polymers obtained by the action of chloroacetic acid or sodium chloroacetate on compounds comprising at least one unit of formula:

-D-X-D-X-D- (VIII)

where D denotes a group

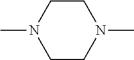

and X denotes the symbol E or E′, E or E′, which may be identical or different, denote a divalent group that is an alkylene group with a straight or branched chain containing up to 7 carbon atoms in the main chain, which is unsubstituted or substituted with hydroxyl groups and which can comprise, in addition to the oxygen, nitrogen and sulfur atoms, 1 to 3 aromatic and/or heterocyclic rings; the oxygen, nitrogen and sulfur atoms being present in the form of ether, thioether, sulfoxide, sulfone, sulfonium, alkylamine or alkenylamine groups, hydroxyl, benzylamine, amine oxide, quaternary ammonium, amide, imide, alcohol, ester and/or urethane groups;
b) polymers of formula:

-D-X-D-X- (IX)

where D denotes a group

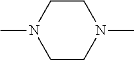

and X denotes the symbol E or E′ and at least once E′; E having the meaning given above and E′ is a divalent group that is an alkylene group with a straight or branched chain having up to 7 carbon atoms in the main chain, which is unsubstituted or substituted with one or more hydroxyl groups and contains one or more nitrogen atoms, the nitrogen atom being substituted with an alkyl chain that is optionally interrupted by an oxygen atom and necessarily comprising one or more carboxyl functions or one or more hydroxyl functions and betainized by reaction with chloroacetic acid or sodium chloroacetate.
(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkylaminoalkanol. These copolymers can also comprise other vinyl comonomers such as vinylcaprolactam.

Among the amphoteric fixing polymers mentioned above, the ones that are most particularly preferred according to the invention are those of family (3), such as the copolymers having the CTFA name octylacrylamide/acrylates/butylaminoethyl methacrylate copolymer, such as the products sold under the names Amphomer®, Amphomer® LV 71 or Lovocryl® 47 by the company Akzo Nobel and those of family (4) such as the copolymers of methyl methacrylate/methyl dimethylcarboxymethylammonioethyl methacrylate, sold, for example, under the name Diaformer Z301 by the company Sandoz.

The nonionic fixing polymers that may be used according to the present invention are chosen, for example, from:
polyalkyloxazolines;
vinyl acetate homopolymers;
vinyl acetate copolymers, for instance copolymers of vinyl acetate and of acrylic ester; copolymers of vinyl acetate and of ethylene, or copolymers of vinyl acetate and of maleic ester, for example of dibutyl maleate;
homopolymers and copolymers of acrylic esters, for instance copolymers of alkyl acrylates and of alkyl methacrylates, such as the products sold by the company Rohm & Haas under the names Primal® AC-261 K and Eudragit® NE 30 D, by the company BASF under the name 8845, or by the company Hoechst under the name Appretan® N9212;
copolymers of acrylonitrile and of a nonionic monomer selected, for example, from butadiene and alkyl (meth)acrylates; mention may be made of the products sold under the name CJ 0601 B by the company Rohm & Haas;
styrene homopolymers;
styrene copolymers, for instance copolymers of styrene and of an alkyl (meth)acrylate, such as the products Mowilith® LDM 6911, Mowilith® DM 611 and Mowilith® LDM 6070 sold by the company Hoechst, and the products Rhodopas® SD 215 and Rhodopas® DS 910 sold by the company Rhône-Poulenc; copolymers of styrene, of alkyl methacrylate and of alkyl acrylate; copolymers of styrene and of butadiene; or copolymers of styrene, of butadiene and of vinylpyridine;
polyamides;
vinyllactam homopolymers such as vinylpyrrolidone homopolymers and such as the polyvinylcaprolactam sold under the name Luviskol® Plus by the company BASF; and
vinyllactam copolymers such as a poly(vinylpyrrolidone/vinyllactam) copolymer sold under the trade name Luvitec® VPC 55K65W by the company BASF, poly(vinylpyrrolidone/vinyl acetate) copolymers, such as those sold under the name PVPVA® S630L by the company ISP, Luviskol® VA 73, VA 64, VA 55, VA 37 and VA 28 by the company BASF; and poly(vinylpyrrolidone/vinyl acetate/vinyl propionate) terpolymers, for instance the product sold under the name Luviskol® VAP 343 by the company BASF.
non-hydrolysed polyvinylformamide polymers, such as the product sold under the name Lupamin 9000 by the company BASF.

The alkyl groups of the abovementioned nonionic polymers preferably have from 1 to 6 carbon atoms.

According to the invention, it is also possible to use fixing polymers of grafted silicone type comprising a polysiloxane portion and a portion constituted of a non-silicone organic chain, one of the two portions constituting the main chain of the polymer and the other being grafted to said main chain.

These polymers are described, for example, in patent applications EP-A-0 412 704, EP-A-0 412 707, EP-A-0 640 105 and WO 95/00578, EP-A-0 582 152 and WO 93/23009 and patents U.S. Pat. No. 4,693,935, U.S. Pat. No. 4,728,571 and U.S. Pat. No. 4,972,037.

These polymers may be amphoteric, anionic or nonionic, and are preferably anionic or nonionic.

Such polymers are, for example, copolymers capable of being obtained by radical polymerization starting from the monomer mixture formed:

a) of 50% to 90% by weight of tert-butyl acrylate,
b) of 0% to 40% by weight of acrylic acid,
c) of 5% to 40% by weight of a silicone macromer of formula:

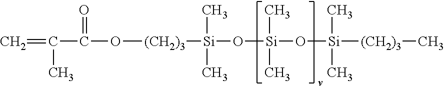

in which v is a number ranging from 5 to 700, the weight percentages being calculated relative to the total weight of the monomers.

Other examples of grafted silicone polymers are in particular polydimethylsiloxanes (PDMSs) to which mixed polymer units of the poly(meth)acrylic acid type and of the poly(alkyl (meth)acrylate) type are grafted via a thiopropylene-type connecting link and polydimethylsiloxanes (PDMSs) to which polymer units of the poly(isobutyl (meth)acrylate) type are grafted via a thiopropylene-type connecting link.

Another type of silicone fixing polymer that may be mentioned is the product Luviflex® Silk, sold by the company BASF.

As fixing polymers it is also possible to use functionalized or unfunctionalized, silicone or non-silicone, cationic, nonionic, anionic or amphoteric polyurethanes, or mixtures thereof.

The polyurethanes particularly targeted by the present invention are those described in patent applications EP 0 751 162, EP 0 637 600, EP 0 648 485 and FR 2 743 297, for which the applicant is the proprietor, and patent applications EP 0 656 021 and WO 94/03510 from the company BASF and EP 0 619 111 from the company Akzo Nobel.

As polyurethanes that are particularly suitable for the present invention, mention may be made of the products sold under the names Luviset Pur® and Luviset® Si Pur by the company BASF.

Preferably, the fixing polymer(s) of the invention is (are) chosen from anionic or nonionic fixing polymers and more preferably still from anionic fixing polymers.

The fixing polymer(s) of the present invention is (are) present in a concentration ranging from 0.1% to 40% by weight, preferably ranging from 0.5% to 25% by weight and more preferably still ranging from 1% to 10% by weight of the total weight of the composition.

The composition may be aqueous or anhydrous. It is preferably aqueous. It then preferably comprises from 5% to 97% of water, more preferably still ranging from 20% to 95% of water, better still from 50% to 90% of water.

The composition may comprise at least one organic solvent preferably chosen from C₁-C₄ lower alcohols, such as ethanol, isopropanol, tert-butanol or n-butanol; polyols, such as glycerol, propylene glycol and polyethylene glycols; acetone, propylene carbonate; benzyl alcohol, glycol ether derivatives; aminoethylpropanol, and mixtures thereof.

The pH of the compositions used according to the invention is generally between 1 and 13, preferably between 3 and 11 and more preferably still between 6 and 9.

Preferably, the composition according to the invention may contain active agents conventionally used in non-long-lasting hair shaping, other than those described previously, and chosen from cationic surfactants, nonionic surfactants other than those of the invention, anionic surfactants, amphoteric surfactants, silicones, non-associative thickeners, cationic associative polymers, direct dyes, in particular cationic or natural direct dyes or oxidation dyes, organic or mineral pigments, UV-screening agents, fillers, in particular nacres, TiO₂, resins or clays, fragrances, peptizers, vitamins, amino acids, preservatives, agents for long-lasting hair shaping, in particular thiolated organic reducing agents, non-thiolated organic reducing agents, alkaline agents, etc.

Of course, a person skilled in the art will take care to choose the optional additional compounds and/or their amounts so that the advantageous properties of the compositions used according to the invention are not, or not substantially, detrimentally affected by the envisaged addition.

Preferably, the compositions are in the form of gels. Preferably, the compositions have a viscosity of greater than or equal to 4 Pa·s and better still ranging from 4 Pa·s to 500 Pa·s at a temperature of 25° C. and at a shear rate of 1 s⁻¹ (measurable, for example, with a Haake RS600 rheometer).

The composition according to the invention may especially be used as a leave-in application on the hair.

Another subject of the present invention is a method for treating keratin fibres such as the hair, comprising the application of a cosmetic composition according to the invention.

The invention is illustrated in more detail in the following examples, which are provided by way of illustration and without implied limitation of the invention.

### EXAMPLES

The following styling gel formulations according to the invention were prepared. The concentrations are expressed as weight percentages of active materials in the final composition.

### Examples 1 to 5

| | Concentrations AM (%) | | | | |
|---|---|---|---|---|---|
| Compounds | 1 | 2 | 3 | 4 | 5 |
| ACRYLATES/STEARETH-20 | 1.95 | 1.95 | 1.95 | 1.95 | 1.95 |
| METHACRYLATE | | | | | |
| CROSSPOLYMER^{(a)} | | | | | |
| ACRYLATES/HYDROXYESTERS | 4.7 | 4.7 | 4.7 | | |
| ACRYLATES COPOLYMER^{(b)} | | | | | |
| VINYLPYRROLIDONE/ | | | | | 4.96 |
| DIMETHYL AMINOETHYL | | | | | |
| METHACRYLATE | | | | | |
| COPOLYMER^{(c)} | | | | | |
| N-VINYLFORMAMIDE^{(d)} | | | | 1.2 | |
| Laureth-4 | | 5 | | | |
| Laureth-6 | | | 5 | | |
| OXYETHYLENATED (20 EO) | 5 | | | | |
| SORBITAN MONOLAURATE | | | | | |
| (POLYSORBATE 20)^{(e)} | | | | | |
| Cocoglucoside^{(f)} | | | | 2.6 | 2.6 |
| Aminomethylpropanol | 1.5 | 1.5 | 1.5 | 1.5 | 1.5 |
| Water | QS | QS | QS | QS | QS |

^{(a)}ACULYN 88 sold by DOW CHEMICAL
^{(b)}ACUDYNE 180 sold by DOW CHEMICAL
^{(c)}COPOLYMER 845-O sold by ISP
^{(d)}LUPAMIN 9000 sold by BASF
^{(e)}Tween 20 sold by CRODA
^{(f)}PLANTACARE 818 UP

### Examples 6 and 7

Concentrations AM (%)

| Compounds | 6 | 7 |
|---|---|---|
| ACRYLATES/STEARETH-20 | 1.35 | 1.35 |
| METHACRYLATE COPOLYMER^{(f)} | | |
| ACRYLATES/HYDROXYESTERS | 4.7 | 4.7 |
| ACRYLATES COPOLYMER^{(g)} | | |
| Laureth-4 | 5 | |
| Laureth-6 | | 5 |
| Aminomethylpropanol | 1 | 1 |
| Water | QS | QS |

^{(f)}ACULYN 22 sold by DOW CHEMICAL
^{(g)}ACUDYNE 180 sold by DOW CHEMICAL

### Examples 8 to 10

| | Concentrations AM (%) | | |
|---|---|---|---|
| Compounds | 8 | 9 | 10 |
| POLYOXYETHYLENATED | 3 | 3 | 3 |
| STEARYL ALPHA, OMEGA- | | | |
| TERMINATED POLYETHER | | | |
| POLYURETHANE^{(h)} | | | |
| ACRYLATES/t- | 8 | 8 | 8 |
| BUTYLACRYLAMIDE | | | |
| COPOLYMER⁽ⁱ⁾ | | | |
| Laureth-4 | 5 | | |
| Laureth-6 | | 5 | |
| Polyglycerolated (3.5 mol) | | | 5 |
| dodecanediol alcohol^{(j)} | | | |
| Aminomethylpropanol | 1 | 1 | 1 |
| Water | QS | QS | QS |

^{(h)}RHEOLATE FX 1100 ® sold by ELEMENTIS
⁽ⁱ⁾ULTRA HOLD STRONG ® sold by DOW CHEMICAL
^{(j)}CHIMEXANE NF (CHIMEX)

### Protocol for Application to 2.7 q Locks of Natural Chestnut-Brown Hair

1. Shampooing with Ultra Doux Camomille shampoo.
2. Rinsing with water, the temperature of the water being 38° C. and the flow rate being 4 l/min, then removal of the shampoo by passing the fingers through 15 times under water.
3. Wringing out the locks by 2 passes between the fingers.
4. Applying 1 g of the formulation homogeneously along the wet lock.
5. Drying in the open air.

For each of the compositions according to the invention, the gel obtained is homogeneous and thick enough to enable quick and easy application of the composition to the hair. In addition, the product slides well in the hands and on the hair so as to obtain uniform distribution over the entire head of hair.

On application and after drying, significant shaping of the hair and hold of this shape are obtained (presence of hardened locks).

### Evaluation of the Fixing Hold:

The hold of the hair is evaluated by successive passes of cold flat tongs along the lock. As more and more passes are performed, the lock becomes less rigid since the polymer film breaks. The more the lock withstands the passes of the flat tongs, the greater the hold of the gel.

It is found that the gels formulated according to the invention allow a markedly greater fixing hold than in the case of the same styling gels without surfactant.

Evaluation of the Rinsing of the Gels from the Hair:

The locks prepared beforehand according to the protocol above are then rinsed with water according to the following protocol:
1. Rinsing with water, the temperature of the water being 38° C. and the flow rate being 4 l/min, followed by passing the fingers through 15 times under water.
2. Wringing out the locks by 2 passes between the fingers.
3. Drying in the open air.

The locks are then classed according to their degree of cleanliness (removal of the product, smooth feel with no residue) in comparison with an untreated lock and relative to the same gel without surfactant.

It is found each time that the locks treated with a styling gel according to the invention are cleaner after rinsing with water than in the case of the same gels without surfactant.

Evaluation of the Rinsing of the Gels from the Hands:

1 gram of gel is applied to the hands. The product is spread between both hands. The hands are then passed under water (water temperature 38° C., flow rate 4 l/min) and the rinsing time is measured.

It is found each time that the hands are rinsed much more quickly with a styling gel according to the invention. They are cleaner after rinsing with water than in the case of the same gel without surfactant.

1-14. (canceled)

15. A cosmetic composition comprising:
at least one associative polymer chosen from anionic and nonionic associative polymers,
at least one fixing polymer, and
at least one nonionic surfactant comprising predominantly fatty chains having from 8 to 14 carbon atoms,
wherein the at least one nonionic surfactant comprising predominantly fatty chains having from 8 to 14 carbon atoms is present in the cosmetic composition in a concentration greater than or equal to about 2% by weight relative to the total weight of the cosmetic composition.

16. The cosmetic composition according to claim 15, wherein the anionic associative polymers are selected from:
polymers comprising at least one hydrophilic unit, and at least one fatty-chain allyl ether unit;
polymers comprising at least one hydrophilic unit of unsaturated olefinic carboxylic acid type, and at least one hydrophobic unit of (C₁₀-C₃₀)alkyl ester of unsaturated carboxylic acid type;
maleic anhydride/C₃₀-C₃₈ α-olefin/alkyl maleate terpolymers;
acrylic terpolymers comprising: (a) 20% to 70% by weight of an α,β-monoethylenically unsaturated carboxylic acid, (b) 20% to 80% by weight of a non-surfactant α,β-monoethylenically unsaturated monomer different from (a), and (c) 0.5% to 60% by weight of a nonionic monourethane which is the reaction product of a monohydric surfactant with a monoethylenically unsaturated monoisocyanate; and
copolymers comprising among their monomers an α,β-monoethylenically unsaturated carboxylic acid and an ester of an α,β-monoethylenically unsaturated carboxylic acid and of an oxyalkylenated fatty alcohol.

17. The cosmetic composition according to claim 16, wherein the anionic associative polymers are selected from polymers in which
the hydrophilic unit of unsaturated olefinic carboxylic acid type corresponds to the monomer of formula (II): 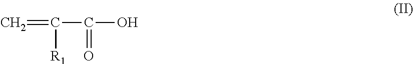
wherein R₁ is chosen from H, CH₃, and C₂H₅, and
the hydrophobic unit of (C₁₀-C₃₀) alkyl ester of unsaturated carboxylic acid type corresponds to the monomer of formula (III): 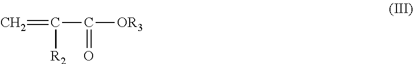
wherein R₂ is chosen from H, CH₃, and C₂H₅, and R₃ is chosen from C₁₀-C₃₀ alkyl radicals.

18. The cosmetic composition according to claim 15, wherein the anionic associative polymers are selected from copolymers comprising among their monomers an α,β-monoethylenically unsaturated carboxylic acid and an ester of an α,β-monoethylenically unsaturated carboxylic acid and of an oxyalkylenated fatty alcohol.

19. The cosmetic composition according to claim 15, wherein the nonionic associative polymers are selected from:
celluloses modified with groups comprising at least one fatty chain;
hydroxypropyl guars modified with groups comprising at least one fatty chain;
inulins modified with groups comprising at least one fatty chain;
copolymers of vinylpyrrolidone and of fatty-chain hydrophobic monomers;
copolymers of C₁-C₆ alkyl methacrylates or acrylates and of amphiphilic monomers comprising at least one fatty chain;
copolymers of hydrophilic methacrylates or acrylates and of hydrophobic monomers comprising at least one fatty chain;
polyether polyurethanes comprising in their chain both hydrophilic blocks of polyoxyethylenated nature and hydrophobic blocks; and
polymers with an aminoplast ether backbone containing at least one fatty chain.

20. The cosmetic composition according to claim 15, wherein the nonionic associative polymers are selected from polyether polyurethanes comprising in their chain both hydrophilic blocks of polyoxyethylenated nature and hydrophobic blocks.

21. The cosmetic composition according to claim 15, wherein the at least one associative polymer is present in the cosmetic composition in a concentration ranging from about 0.01% to about 30% by weight relative to the total weight of the cosmetic composition.

22. The cosmetic composition according to claim 21, wherein the at least one associative polymer is present in the cosmetic composition in a concentration ranging from about 0.5% to about 10% by weight relative to the total weight of the cosmetic composition.

23. The cosmetic composition according to claim 15, wherein the at least one fixing polymer is selected from anionic, cationic, nonionic and amphoteric fixing polymers.

24. The cosmetic composition according to claim 15, wherein the at least one fixing polymer is chosen from anionic and cationic fixing polymers.

25. The cosmetic composition according to claim 24, wherein the at least one fixing polymer is selected from:
copolymers of acrylic or methacrylic acid;
copolymers of crotonic acid;
copolymers of C₄-C₈ monounsaturated carboxylic acids or anhydrides;
polyacrylamides comprising carboxylate groups; and
homopolymers and copolymers comprising sulfonic groups such as polymers comprising vinylsulfonic, styrenesulfonic, naphthalenesulfonic or acrylamidoalkylsulfonic units.

26. The cosmetic composition according to claim 15, wherein the at least one fixing polymer is selected from:
homopolymers or copolymers derived from acrylic or methacrylic esters or amides and comprising at least one of the units of the following formulae: 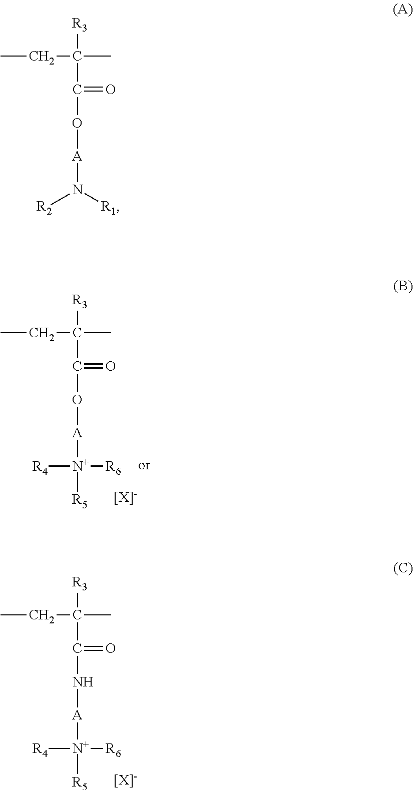
wherein:
R₃ is chosen from hydrogen and CH₃ radicals;
A is chosen from linear or branched alkyl groups comprising from 1 to 6 carbon atoms and hydroxyalkyl groups comprising from 1 to 4 carbon atoms;
R₄, R₅ and R₆, which may be identical or different, are chosen from alkyl groups comprising from 1 to 18 carbon atoms and benzyl radicals;
R₁ and R₂, which may be identical or different, are chosen from hydrogen and alkyl groups comprising from 1 to 6 carbon atoms; and
X denotes a methosulfate anion or a halide such as chloride or bromide;
non-cellulose cationic polysaccharides;
quaternary copolymers of vinylpyrrolidone and of vinylimidazole;
chitosans and salts thereof;
cellulose derivatives; and
partially hydrolysed polyvinylformamide polymers.

27. The cosmetic composition according to claim 15, wherein the at least one fixing polymer is present in the cosmetic composition in a concentration ranging from about 0.1% to about 40% by weight relative to the total weight of the cosmetic composition.

28. The cosmetic composition according to claim 27, wherein the at least one fixing polymer is present in the cosmetic composition in a concentration ranging from about 1% to about 10% by weight relative to the total weight of the cosmetic composition.

29. The cosmetic composition according to claim 15, wherein the at least one nonionic surfactant comprising predominantly fatty chains having from 8 to 14 carbon atoms is selected from oxyethylenated C₈-C₁₄ alcohols comprising from 1 to 100 mol of ethylene oxide, polyoxyethylenated esters of linear or branched, saturated or unsaturated C₈-C₁₄ acids and of sorbitol comprising from 1 to 100 mol of ethylene oxide, C₈-C₁₄ alkyl polyglycosides, and monoglycerolated or polyglycerolated nonionic surfactants of C₈-C₁₄ alcohols, which are optionally hydroxylated.

30. The cosmetic composition according to claim 15, wherein the at least one nonionic surfactant comprising predominantly fatty chains having from 8 to 14 carbon atoms is present in the cosmetic composition in a concentration ranging from about 2% to about 40% by weight relative to the total weight of the cosmetic composition.

31. The cosmetic composition according to claim 30, wherein the at least one nonionic surfactant comprising predominantly fatty chains having from 8 to 14 carbon atoms is present in the cosmetic composition in a concentration ranging from about 4% to about 10% by weight relative to the total weight of the cosmetic composition.

32. A method for the cosmetic treatment of keratin fibers, comprising applying thereto a cosmetic composition comprising:
at least one associative polymer chosen from anionic and nonionic associative polymers,
at least one fixing polymer, and
at least one nonionic surfactant comprising predominantly fatty chains having from 8 to 14 carbon atoms,
wherein the at least one nonionic surfactant comprising predominantly fatty chains having from 8 to 14 carbon atoms is present in the cosmetic composition in a concentration greater than or equal to about 2% by weight relative to the total weight of the cosmetic composition.

33. The method according to claim 32, wherein the cosmetic composition is applied to the hair.

34. The method according to claim 33, wherein the cosmetic treatment comprises form retention and/or fixing of the hair.
